(19) United States
(12) Patent Application Publication
(10) Pub. No.: US 2019/0328362 A1
(43) Pub. Date: Oct. 31, 2019

(54) WIRE ASSEMBLY WITH ECHOGENIC FEATURES AND METHOD OF FABRICATING WIRE ASSEMBLY WITH ECHOGENIC FEATURES

(71) Applicant: Spiration, Inc. d/b/a Olympus Respiratory America, Redmond, WA (US)

(72) Inventor: Peter A. LAMBE, Redmond, WA (US)

(73) Assignee: Spiration, Inc. d/b/a Olympus Respiratory America, Redmond, WA (US)

(21) Appl. No.: 16/476,052

(22) PCT Filed: Jan. 4, 2017
(86) PCT No.: PCT/US2017/012225

§ 371 Date: Jul. 3, 2019

(51) Int. Cl. A61B 8/08; A61B 18/14; A61B 34/20

(52) U.S. Cl. CPC A61B 8/481; A61B 18/14; A61B 2018/00577; A61B 8/0841; A61B 34/20

(57) ABSTRACT

Disclosed embodiments include a wire assembly having echogenic features, an electrosurgical device, a system for treating tissue, a method for treating tissue, and a method for fabricating a wire assembly having echogenic features. In an illustrative, non-limiting embodiment, a wire assembly includes: a wire having a finite length and an exterior surface, at least a portion of the wire having at least one depression defined in the exterior surface; and tubing disposed in an airtight manner over the exterior surface of the at least a portion of the wire having the at least one depression defined in the exterior surface, the tubing and the at least one depression forming an air pocket therebetween.

### FIELD

The present disclosure relates to a wire assembly with echogenic features.

### BACKGROUND

The statements in this section merely provide background information related to the present disclosure and may not constitute prior art.

Ultrasound imaging is used for visualization in many applications, such as without limitation in-vivo imaging of various electrosurgical instruments. For example, certain electrosurgical instruments used for treating tissue generally may include a guide catheter and an applicator inserted through the catheter. These electrosurgical instruments may be inserted into a body lumen to place the distal end of the applicator at a desired location.

The applicator generally includes one or more electrodes at the distal end. Such electrodes may emit a radiofrequency (“RF”) electric current to surrounding tissue to coagulate and/or ablate the tissue. Monopolar electrosurgical instruments only entail use of one electrode that interacts with a neutral electrode, which is likewise connected to the body of a patient. A bipolar electrosurgical instrument typically includes an applicator with two electrodes (that is, a distal electrode and a proximal electrode). An RF voltage with different potentials is applied to such bipolar instruments so that a current passes from one electrode to the other electrode through the tissue, thereby heating the tissue to coagulate or ablate the tissue.

During the procedure, a sensor (such as an ultrasound transducer, a visual camera, and the like) is used at an end of the catheter to view the applicator's location relative to target tissue. However, the applicator may be difficult to see in images (such as ultrasound video) and a range of angles in which the wire may be seen may be limited in cases where the applicator may include electrodes made of thin wires (such as those on the order of around 20/1000 inch) that may have insufficient echogenic properties.

### SUMMARY

Disclosed embodiments include a wire assembly having echogenic features, an electrosurgical device, a system for treating tissue, a method for treating tissue, and a method for fabricating a wire assembly having echogenic features.

In an illustrative embodiment, a wire assembly includes: a wire having a finite length and an exterior surface, at least a portion of the wire having at least one depression defined in the exterior surface; and tubing disposed in an airtight manner over the exterior surface of the at least a portion of the wire having the at least one depression defined in the exterior surface, the tubing and the at least one depression forming an air pocket therebetween.

In another illustrative embodiment, an electrosurgical device includes: a needle configured as a first electrode; a flat wire coil extendable through the needle and configured as a second electrode, the flat wire coil being movable relative to the needle and insertable into target tissue, at least a first portion of the flat wire coil being coilable and twistable, at least a second portion of the wire having at least one depression defined in the exterior surface; and tubing disposed in an airtight manner over the exterior surface of the at least a second portion of the wire having the at least one depression defined in the exterior surface, the tubing and the at least one depression forming an air pocket therebetween.

In another illustrative embodiment, a system for treating tissue includes: a source of electrical power; a needle electrically coupled to the source of electrical power and configured as a first electrode; a flat wire coil electrically coupled to the source of electrical power, the flat wire coil being extendable through the needle and configured as a second electrode, the flat wire coil being movable relative to the needle and insertable into target tissue, at least a first portion of the flat wire coil being coilable and twistable, at least a second portion of the wire having at least one depression defined in the exterior surface; and tubing disposed in an airtight manner over the exterior surface of the at least a second portion of the wire having the at least one depression defined in the exterior surface, the tubing and the at least one depression forming an air pocket therebetween.

In another illustrative embodiment, a method for treating tissue includes: positioning an applicator in a passageway; extending a needle through the applicator, the needle being a first electrode; piercing the needle into target tissue; advancing a flat wire coil through the needle, a distal portion of the flat wire coil piercing into the target tissue, at least a first portion of the flat wire coil attaining a coiled configuration and a twisted configuration as the flat wire coil is inserted into the target tissue, at least a second portion of the flat wire coil having at least one depression defined in an exterior surface thereof, the at least a second portion of the flat wire coil having tubing disposed in an airtight manner over an exterior surface thereof, the tubing and the at least one depression forming an air pocket therebetween; and ultrasonically illuminating the flat wire coil.

In another illustrative embodiment, a method for fabricating a wire assembly having echogenic features includes: defining at least one depression in an exterior surface of at least a portion of a wire; and disposing tubing in an airtight manner over the exterior surface of the at least a portion of the wire having the at least one depression defined in the exterior surface, the tubing and the at least one depression forming an air pocket therebetween.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, with emphasis instead being placed upon illustrating the principles of the disclosed embodiments. In the drawings:

FIG. 1 is a perspective view in partial cutaway of an illustrative wire assembly having echogenic features.

FIG. 2 is an end plan view in cutaway of the wire assembly of FIG. 1.

FIGS. 3A-3E illustrate non-limiting patterns of echogenic features of the wire assembly of FIG. 1.

FIG. 4 is a block diagram in partial schematic form of an illustrative system for treating tissue;

FIG. 5 is a side plan view in partial schematic form of an illustrative distal end of an insertion device of the system of FIG. 4;

FIG. 6 illustrates the device of FIG. 5 during a first operational step; and

FIGS. 7 and 8 illustrate the device of FIG. 5 during a second operational step.

### DETAILED DESCRIPTION

The following description is merely exemplary in nature and is not intended to limit the present disclosure, application, or uses.

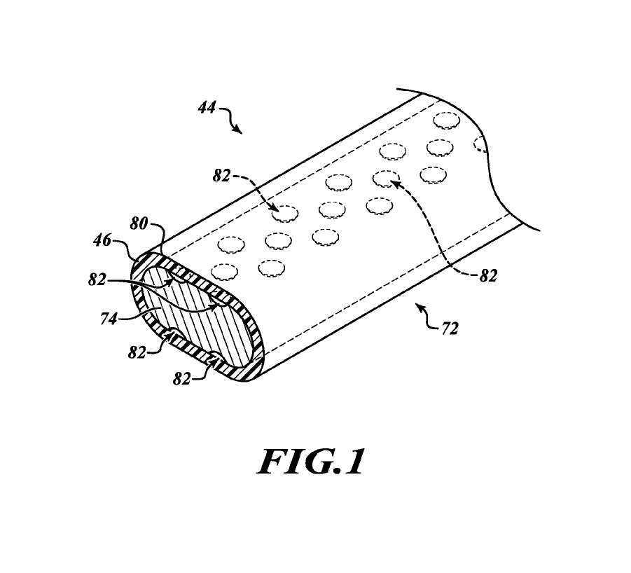

Given by way of overview and referring to FIG. 1, in an illustrative, non-limiting embodiment, a wire assembly 44 includes a wire 74 having a finite length and an exterior surface 80. At least a portion 72 of the wire 74 has at least one depression 82 defined in the exterior surface 80. Tubing 46 is disposed in an airtight manner over the exterior surface 80 of the portion 72 of the wire 74 that has the at least one depression 82 defined in the exterior surface 80. The tubing 46 and the depression 82 form an air pocket therebetween. The air pocket formed between the tubing 46 and the depression 82 provides an echogenic feature that may help provide enhanced ultrasound returns from the wire assembly 44 due to a large difference in density between density of air and density of materials used for the wire 74, thereby helping to enhance ultrasound illumination of the wire assembly 44.

Now that an overview has been provided, details will be set forth below by way of non-limiting examples and not of limitation.

Referring additionally to FIG. 2, in some embodiments and given by way of non-limiting example, the wire 74 may be a flat wire. In such embodiments, the wire 74 has four sides 84, 86, 88, and 90. Two opposing sides 84 and 86 may have a width w₁ that is greater than than a width w₂ of the other opposing sides 88 and 90. In some embodiments such as without limitation embodiments with electrosurgical applications, the wire 74 may be thin. For example, in such embodiments the width w₂ may be on the order of around 20/1000 inch or so. In such embodiments, it will be appreciated that any enhancement of ultrasound illumination of the wire assembly 44 by virtue of enhanced ultrasound returns from the wire assembly 44 due to echogenicity of the air pocket formed between the tubing 46 and the depression 82 may help to increase a range of angles through which the wire assembly 44 can be seen via ultrasound illumination (compared to conventional, thin wire assemblies without echogenic features).

The wire 74 suitably is made from any material as desired for a particular application. For example and without limitation, in various embodiments the wire 74 may be made from copper, aluminum, steel, silver, aluminum, and the like. In some embodiments such as without limitation those with electrosurgical applications, the wire 74 may be made from any suitable material, such as stainless steel, that enables the wire 74 to be corkscrewed into tissue.

In various embodiments, all or a portion of the wire 74 may be made from a shape memory alloy (also known as smart metal, memory metal, memory alloy, muscle wire, and smart alloy) for its super-elastic properties and/or its shape memory features. A suitable shape memory alloy may include, without limitation, nitinol (NiTi), copper-aluminum-nickel alloys, copper-zinc-aluminum alloys, iron-manganese-silicon alloys, and the like, as desired for a particular application. Given by way of non-limiting example and as will be discussed below, in embodiments that entail an electrosurgical application the wire 74 may include at least a portion that is made of the shape memory alloy nitinol. When the wire 74 is made of shape memory alloy and is implemented for its shape memory properties, the portion of the wire 74 made of shape memory alloy may have multiple configurations or states. Accordingly, when the wire 74 is in one of the configurations and then heated, the wire 74 returns to another pre-defined configuration. Subsequently, if the wire 74 is cooled, the wire 74 returns to the configuration it had when unheated. When a portion of the wire 74 is made of shape memory alloy and other portions of the wire 74 are made of other materials (such as those discussed above), the depressions 82 may be located in the portion made of shape memory alloy, if desired, and may be located in the other portions made of other materials, as desired for a particular application.

The depressions 82 may be formed in any shape as desired. For example and without limitation, the depressions 82 may have cross-sectional shapes that are circular, elliptical, ovoid, random, or the like. The depressions 82 may have any suitable size as desired for a particular application and as appropriate for size of the wire 74. For example and given by way of illustration and not of limitation, in some embodiments with electrosurgical applications the wire 74 may have a thickness on the order of around 20/1000 inch. In such embodiments, the depressions 82 may have a depth of around 1/1000 inch and may have a cross-sectional dimension on the order of around 4/1000 inch. It will be appreciated that such dimensions are illustrative only and are not limiting in any manner whatsoever.

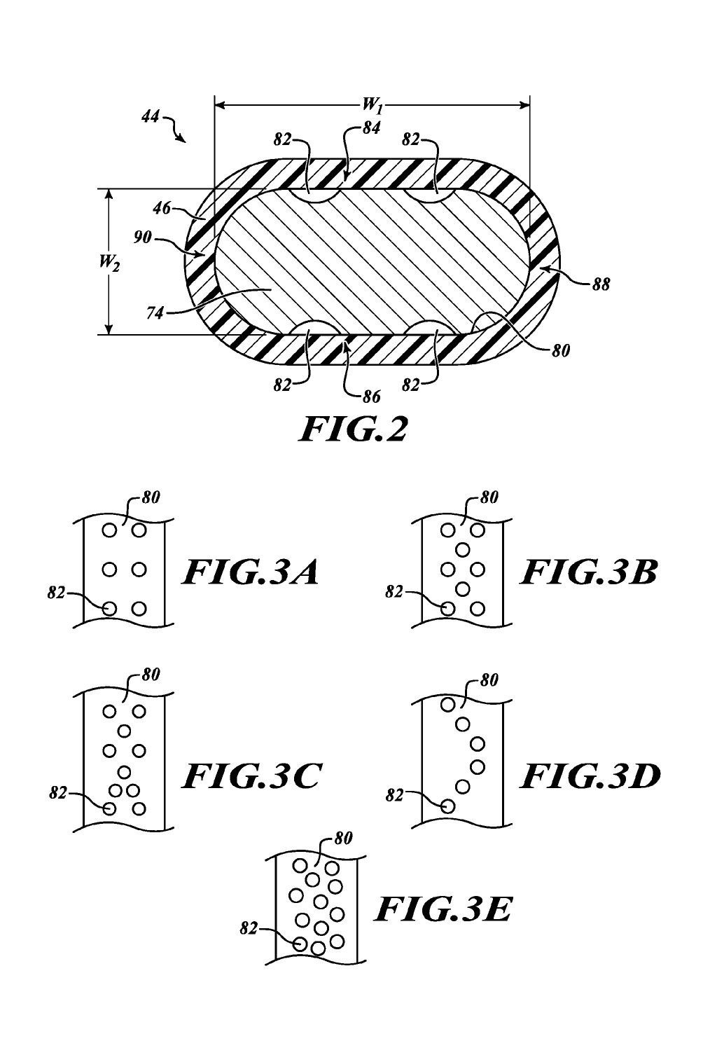

Referring additionally to FIGS. 3A-3E, the depressions 82 may be located in the exterior surface 80 as desired. As shown in FIGS. 3A-3D, the depressions 82 may form a pattern that may be repeatable. As shown in FIG. 3E, the depressions 82 may be located in the exterior surface 82 in a random manner. It will be appreciated that the depressions 82 shown in FIGS. 3A-3E are illustrative only and that no limiting inferences regarding shape of the depressions 82 are to be made.

The depressions 82 may be formed by any suitable method as desired. For example and without limitation, the depressions 82 may be formed by processes such as without limitation laser etching, acid etching, shot peening, abrasive blasting, water blasting, and the like. It will be appreciated that the process to form the depressions 82 may be selected, in part, based upon factors such as compatibility of the process and/or process reactants and/or blasting media, as applicable, with material from which the wire 74 is made, ability to make and/or repeat patterns such as those shown in FIGS. 3A-3D, and the like. For example and without limitation, shot peening or abrasive blasting may be selected as a process to form the depressions 82 when the depressions 82 are to be located in a random manner as shown in FIG. 3E.

The tubing 46 performs two functions. As discussed above, firstly the tubing 46 forms an airtight seal around the portions of the wire 74 that includes the depressions 82 and cooperates with the depressions 82 to form air pockets between the exterior surface 80 of the depressions 82 and the tubing 46. Secondly, the tubing 46 is an electrical insulator. With the two functions discussed above in mind, in various embodiments the tubing 46 includes heat-shrink tubing. The heat shrink tubing may include polyester, such as without limitation polyethylene terephthalate (“PET”) and the like. In some embodiments with electrosurgical applications, the heat-shrink tubing may have a thickness on the order of around 1/1000 inch or so. However, it will be appreciated that the tubing 46 may have any thickness as desired for a particular application.

In various embodiments the depressions 82 are sized (as discussed above) such that the tubing 46 remains out of contact with the exterior surface 80 of the depressions 82. In embodiments in which the tubing 46 includes heat-shrink tubing, the heat-shrink tubing shrinks to the exterior surface 80 of the wire 74 but does not shrink into the depressions 82. Thus, air is trapped in the void (that is, the depression 82) between the tubing 46 and the exterior surface 80 of the depression 82, thereby forming an air pocket. As also discussed above, the large difference in density between density of material from which the wire 74 is made and density of air in the air pocket increases echogenicity of the wire assembly 44 and can help enhance ultrasound illumination of the wire assembly 44.

Embodiments of the wire assembly 44 may be made by any suitable process as desired for a particular application. Given by way of illustration and not of limitation, in various embodiments the wire assembly 44 may be made according to the following illustrative method that is given by way of example only and not of limitation. At least one depression 82 is defined in an exterior surface 80 of at least a portion of a wire 74. Tubing 46 is disposed in an airtight manner over the exterior surface 80 of the at least a portion of the wire 74 having the at least one depression 82 defined in the exterior surface 80, and the tubing 46 and the at least one depression 82 form an air pocket therebetween.

In some embodiments the at least one depression 82 may be defined in the exterior surface 80 of at least a portion of the wire 74 via a process such as laser etching, acid etching, shot peening, abrasive blasting, water blasting, and the like.

In some embodiments the at least one depression 82 is sized such that the tubing 46 remains out of contact with the exterior surface 80 of the at least one depression 82.

It will be appreciated that the wire assembly 44 may be used for any purpose as desired. It will also be appreciated that some embodiments of the wire assembly may be used in electrosurgical settings. For example and without limitation, in some such embodiments the wire assembly 44 may be used as an electrode in an electrosurgical system. An illustrative system environment for an electrosurgical application of a non-limiting embodiment of the wire assembly 44 is set forth below by way of illustration and not of limitation. In the example set forth below, the wire assembly 44 is referred to as a coil 44 and the tubing 46 is referred to as a layer of insulation 46.

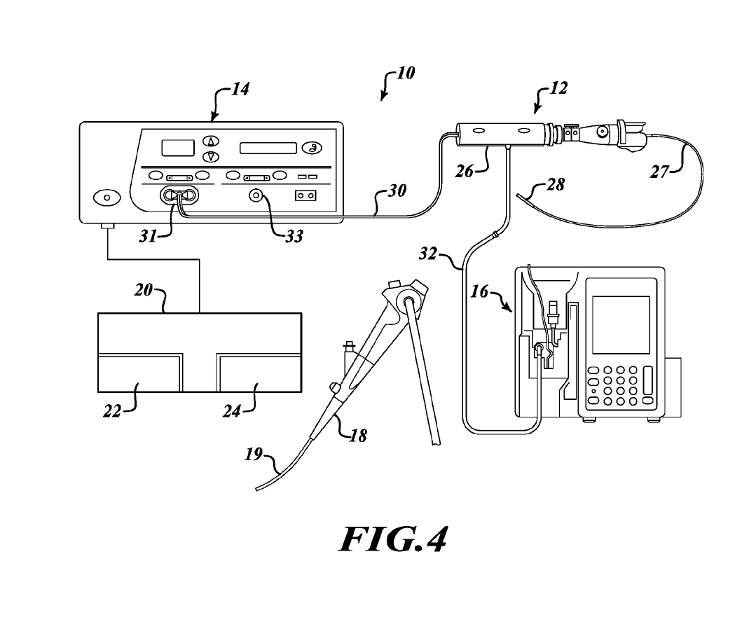

Referring additionally to FIG. 4, a system 10 is provided for treating tissue in an anatomical region of a patient. The system 10 may be a bipolar or monopolar radio frequency (RF) system, as desired, for treating tissue in a patient. Specifically, the system 10 may be employed for coagulation and/or ablation of soft tissue during percutaneous and/or endoscopic, including bronchoscopic, surgical procedures, such as, for example, partial and/or complete ablation of cancerous and/or noncancerous organ lesions.

In some embodiments, the system 10 includes an applicator 12, an electrosurgical RF generator 14, an infusion pump 16, and a bronchoscope 18. The applicator 12 electrically communicates with the generator 14 though a lead 30. In some embodiments, the lead 30 is connected to a generator outlet 31 when the system is operated in a bipolar mode. In some other embodiments, the system 10 can be operated in a monopolar mode when the lead 30 is connected to an outlet 33 with an adapter as desired. The applicator 12 is further connected to the infusion pump 16 with a tube 32 that facilitates the flow of liquid, for example saline solution, from the pump 16 to the applicator 12.

The generator 14 can be operated with the use of a foot operated unit 20 electrically connected to the generator 14. The foot operated unit 20 includes a pedal 22 that instructs the generator 14 to apply an RF potential to electrode(s) (described below) to cut and/or ablate tissue and a pedal 24 that instructs the generator 14 to apply a lower RF potential to the electrode(s) to coagulate tissue.

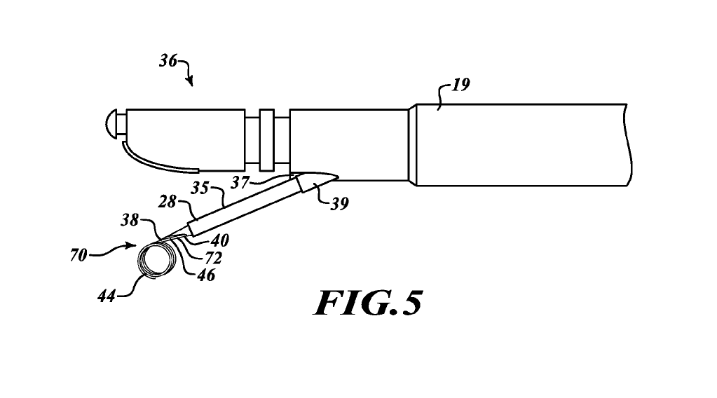

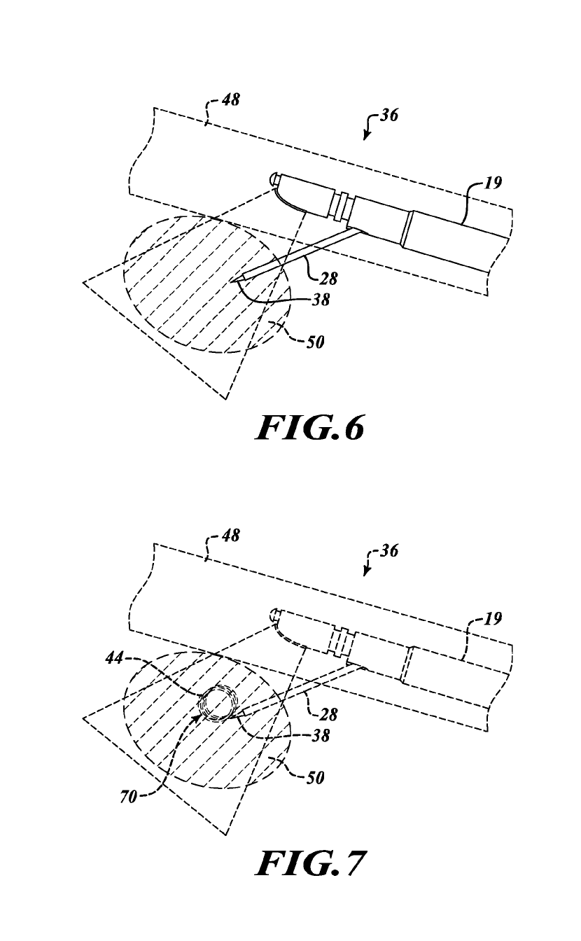

In various embodiments the bronchoscope 18 includes an insertion tube 19. At a distal end 36 (FIG. 5) of the insertion tube 19 is an opening 37 that is proximal to an imaging camera (for example, endobronchial ultrasound (“EBUS”)). The applicator 12 includes a handle 26, a needle 28 and a sheath 27. As such, in certain procedures, the needle 28 and the sheath 27 are inserted into the bronchoscope 18 such that the needle 28 exits the distal end 36 of the insertion tube 19 via the opening 37.

Referring additionally to FIG. 5, in various embodiments the applicator 12 further includes the coil 44 that extends through the sheath 27 and the needle 28 and exits an opening 40 of the needle 28. In various embodiments the needle 28 is made from hypotube, for example, stainless steel. The needle 28 includes a layer of insulation 35. The layer of insulation 35 starts at a location within the sheath 27 and extends to a position proximal to a needle tip 38. The layer of insulation 35 helps keep the covered portions of the needle 28 from generating an external electric field.

The coil 44 includes a coiled portion 70, a non-coiled portion 72, and a layer of insulation 46 that covers the non-coiled portion 72 of the coil 44 to help electrically isolate the coil 44 from the needle 28. The layer of insulation 46 extends to the applicator 12. Accordingly, in this arrangement, the needle 28 operates as a proximal electrode and the coil 44 operates as a distal electrode when the system 10 is operated in a bipolar mode. The non-coiled portion 72 of the coil 44 includes the depressions 82 (not shown in FIG. 5 for clarity purposes) as discussed above. If desired, the coiled portion 70 of the coil 44 may also include the depressions 82 (not shown in FIG. 5 for clarity purposes).

The tip 38 is used for piercing tissue and may include one or more echogenic features. During the penetration of the needle 28 into tissue, only the needle 28 (that is, not the coil 44) is energized in a monopolar mode (for example, with the patient grounded to a patient pad to complete the circuit) with the generator 14 at a first power level. If a penetration force exceeds that which is expected by the physician, then the energized needle tip 38 causes tissue vibration so that it can be visualized ultrasonically. The echogenic features further enhance the ultrasonic visualization of the needle 28.

Referring additionally to FIGS. 6, 7, and 8, a procedure is illustrated for using the system 10 during, for example, bronchoscopy. Initially, a physician advances the insertion tube 19 of the bronchoscope 18 through a passageway, for example, an airway 48, until the distal end 36 is positioned near the desired tissue 50 (for example, a tumor or lesion) to be treated. The physician then inserts the needle 28 into the insertion tube 19 and advances the needle 28 until the needle 28 exits the opening 37 at the distal end 36 and penetrates into the tissue 50 with the tip 38. While being visualized ultrasonically as described above, the needle 28 is positioned at a desired location in the tissue 50 (FIG. 6). Next the physician advances the coil 44 through the needle 28 until it exits the opening 40 to form a coil shape in the tissue 50. Either before, during, or after the coil 44 advances, the coil 44 becomes twisted about its centerline. The physician continues to advance the coil 44 to the desired location (as shown in FIG. 7). Placement of the coil 44 can be visualized ultrasonically due to the twists in the coil 44 and due to echogenicity of the air pockets defined between the depressions 82 and the layer of insulation 46.

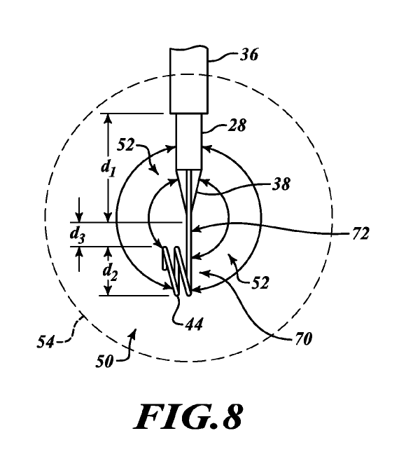

As shown in FIG. 8, the coil 44 corkscrews into the tissue 50 with a diameter d₂. The extent of penetration of the needle 28 (that is, the distance from the opening 37 of the distal end 36 of the insertion tube 19 to the tip 38 of the needle 28) is d₁ and the distance from the coiled portion of the coil 44 to the tip 38 of the needle 28 is d₃. After the coil 44 has been deployed and prior to activating the electrodes (that is, the needle 28 and the coil 44), the needle 28 is retracted proximally while the coil 44 and the insertion tube 19 (and hence the distal end 36) are held in place so that d₁ decreases and d₃ increases.

To energize the electrodes (that is, the needle 28 and the coil 44) for coagulating the tissue 50, the physician sets the generator 14 to a desired second power level and pushes the pedal 24 of the foot unit 20 to apply an RF potential to the electrodes. The second power level is greater than the first power level. As such, RF electrical current passes between the needle 28 and the coil 44 through the tissue 50 as indicated by the arrows 52. The level of RF electrical current is set by the physician to control the desired extent of the coagulation region 54 in the tissue 50. It will be appreciated that that, anytime during the procedure, the physician can activate the infusion pump 16 to supply saline solution to the applicator 12 so that the saline solution flows through the needle 28 and the sheath 27 to the location of interest in the tissue 50. The saline solution is employed to cool the electrodes (that is, the needle 28 and/or the coil 44) and to prevent dehydration of the tissue 50.

In embodiments in which at least a portion of the wire 74 is made of shape memory alloy, the wire 74 is shape set to attain coiled and/or twisted shapes when reaching a predetermined temperature. The predetermined temperature is the austenite finish temperature for the wire 74. Because of the twisted configuration, the flat surface of the wire 74 includes a normal vector that will have different angular relationships with an ultrasound illumination device located at the distal end 36 of the insertion tube 19. In some orientations of the flat surface, the normal vector will be perpendicular or nearly perpendicular to an ultrasound signal produced by the ultrasound illumination device, thereby helping to produce significant ultrasound feedback. Significant ultrasound feedback may occur over the length of the non-coiled portion 72 portion of the coil 44, depending upon the number of depressions 82 in the coil 44. As a result, the non-coiled portion 72 of the coil 44 may produce a more pronounced ultrasonic image than would a straight portion without trapped air pockets. It will be appreciated that a similarly pronounced ultrasonic image may be produced by the coiled portion 70 of the coil 44 in embodiments in which air pockets are provided therein.

After treatment of the tissue 50 is completed, the physician turns off the generator 14 and moves the needle 28 forward to the position prior to deployment of the coil 44. The coil 44 is then retracted into the needle 28. The needle 28 and the coil 44 are then retracted into the insertion tube 19 within the bronchoscope 18, and the bronchoscope 18 is withdrawn from the patient.

It will be appreciated that the detailed description set forth above is merely illustrative in nature and variations that do not depart from the gist and/or spirit of the claimed subject matter are intended to be within the scope of the claims. Such variations are not to be regarded as a departure from the spirit and scope of the claimed subject matter.

What is claimed is:

1. A wire assembly comprising:
a wire having a finite length and an exterior surface, at least a portion of the wire having at least one depression defined in the exterior surface; and
tubing disposed in an airtight manner over the exterior surface of the at least a portion of the wire having the at least one depression defined in the exterior surface, the tubing and the at least one depression forming an air pocket therebetween.

2. The wire assembly of claim 1, wherein the tubing includes heat-shrink tubing.

3. The wire assembly of claim 1, wherein the at least one depression is sized such that the tubing remains out of contact with the exterior surface of the at least one depression.

4. The wire assembly of claim 1, wherein the wire is made from at least one material chosen from copper, aluminum, steel, silver, aluminum, stainless steel, and a shape memory alloy.

5. An electrosurgical device comprising:
a needle configured as a first electrode;
a flat wire coil extendable through the needle and configured as a second electrode, the flat wire coil being movable relative to the needle and insertable into target tissue, at least a first portion of the flat wire coil being coilable and twistable, at least a second portion of the wire having at least one depression defined in the exterior surface; and
tubing disposed in an airtight manner over the exterior surface of the at least a second portion of the wire having the at least one depression defined in the exterior surface, the tubing and the at least one depression forming an air pocket therebetween.

6. The electrosurgical device of claim 5, wherein the tubing includes heat-shrink tubing.

7. The apparatus of claim 5, wherein the at least one depression is sized such that the tubing remains out of contact with the exterior surface of the at least one depression.

8. The electrosurgical device of claim 5, wherein the coil is electrically insulated from the needle.

9. The electrosurgical device of claim 5, wherein the flat wire coil is made from at least one material chosen from steel, stainless steel, and a shape memory alloy.

10. A system for treating tissue, the system comprising:
a source of electrical power;
a needle electrically coupled to the source of electrical power and configured as a first electrode;
a flat wire coil electrically coupled to the source of electrical power, the flat wire coil being extendable through the needle and configured as a second electrode, the flat wire coil being movable relative to the needle and insertable into target tissue, at least a first portion of the flat wire coil being coilable and twistable, at least a second portion of the wire having at least one depression defined in the exterior surface; and
tubing disposed in an airtight manner over the exterior surface of the at least a second portion of the wire having the at least one depression defined in the exterior surface, the tubing and the at least one depression forming an air pocket therebetween.

11. The system of claim 10, further comprising:
an ultrasound device configured to illuminate at least one object chosen from the needle and the flat wire coil and further configured to generate an image based on the illumination.

12. The system of claim 11, wherein the flat wire coil is configured to reflect an ultrasound signal produced by the ultrasound device due to at least one factor chosen from echogenicty of the air pocket and a twisted configuration of the flat wire coil when inserted into the target tissue.

13. The system of claim 10, wherein the flat wire coil is electrically insulated from the needle.

14. A method for treating tissue, the method comprising:
positioning an applicator in a passageway;
extending a needle through the applicator, the needle being a first electrode;
piercing the needle into target tissue;
advancing a flat wire coil through the needle, a distal portion of the flat wire coil piercing into the target tissue, at least a first portion of the flat wire coil attaining a coiled configuration and a twisted configuration as the flat wire coil is inserted into the target tissue, at least a second portion of the flat wire coil having at least one depression defined in an exterior surface thereof, the at least a second portion of the flat wire coil having tubing disposed in an airtight manner over an exterior surface thereof, the tubing and the at least one depression forming an air pocket therebetween; and
ultrasonically illuminating the flat wire coil.

15. The method of claim 14, further comprising:
reflecting an ultrasound signal by the air pocket in the at least a second portion of the flat wire coil.

16. The method of claim 14, further comprising:
reflecting an ultrasound signal by the flat wire coil due to the twisted configuration of the flat wire coil.

17. The method of claim 14, further comprising:
generating an image based on the ultrasonic illuminating.

18. A method for fabricating a wire assembly having echogenic features, the method comprising:
defining at least one depression in an exterior surface of at least a portion of a wire; and
disposing tubing in an airtight manner over the exterior surface of the at least a portion of the wire having the at least one depression defined in the exterior surface, the tubing and the at least one depression forming an air pocket therebetween.

19. The method of claim 18, wherein defining at least one depression in an exterior surface of at least a portion of a wire is performed via a process chosen from laser etching, acid etching, shot peening, abrasive blasting, and water blasting.

20. The method of claim 18, further comprising:
sizing the at least one depression such that the tubing remains out of contact with the exterior surface of the at least one depression.
